SEQUENCE LISTING (20 sequences)

SEQ ID NO: 1 - DNA, 21 bp, Rattus norvegicus
cctctttcccttaccctcctg

SEQ ID NO: 2 - DNA, 20 bp, Homo sapiens
gtcagccatggtcccccccc

SEQ ID NO: 3 - DNA, 21 bp, Rattus norvegicus
cttgagctcagctccctcagg

SEQ ID NO: 4 - DNA, 21 bp, Rattus norvegicus
cctattccctttcctcccaaa

SEQ ID NO: 5 - DNA, 21 bp, Rattus norvegicus
tccactcccccgatccactca

SEQ ID NO: 6 - DNA, 21 bp, Homo sapiens
aacccttatttgtgtcccacc

SEQ ID NO: 7 - DNA, 20 bp, Homo sapiens
cccccaccacttcccctctc

SEQ ID NO: 8 - DNA, 20 bp, Homo sapiens
cggtccccttcaagatccat

SEQ ID NO: 9 - DNA, 20 bp, Homo sapiens
tgcttaccctcccacagcag

SEQ ID NO: 10 - DNA, 21 bp, Rattus norvegicus
agagccacaattccctttcta

SEQ ID NO: 11 - DNA, 14 bp, Homo sapiens
cgaggccaccactc

SEQ ID NO: 12 - DNA, 15 bp, Homo sapiens
ccaccactcatctcg

SEQ ID NO: 13 - DNA, 21 bp, Homo sapiens
cccccaccacttcccctctca

SEQ ID NO: 14 - DNA, 20 bp, Homo sapiens
cattccatgaactctgcaag

SEQ ID NO: 15 - DNA, 21 bp, Rattus norvegicus
cccttaggtttcccagcaagc

SEQ ID NO: 16 - DNA, 15 bp, Homo sapiens
tttgtgtcccacctg

SEQ ID NO: 17 - DNA, 21 bp, Rattus norvegicus
tgatccactcccccctccact

SEQ ID NO: 18 - DNA, 20 bp, Mus musculus
tgccagtccacatagtgttt

SEQ ID NO: 19 - DNA, 21 bp, Rattus norvegicus
tgatccactcccccctccact

SEQ ID NO: 20 - DNA, 20 bp, Homo sapiens
aacccagtgctccctttgct

(19) United States
(12) Patent Application Publication
(10) Pub. No.: US 2004/0073376 A1
(43) Pub. Date: Apr. 15, 2004

(54) Finding active antisense oligonucleotides using artificial neural networks

(75) Inventors: Raymond F. Gesteland, Salt Lake City, UT (US); John F. Atkins, Salt Lake City, UT (US); Olga V. Matveeva, Salt lake City, UT (US); Michael C. Giddings, Chapel Hill, NC (US)

Correspondence Address: ALAN J. HOWARTH, P.O. BOX 1909, SANDY, UT, 84091-1909, US

(73) Assignee: University of Utah Research Foundation

(21) Appl. No.: 10/050,888

(22) Filed: Jan. 14, 2002

Related U.S. Application Data
(60) Provisional application No. 60/262,993, filed on Jan. 19, 2001.

(51) Int. Cl. G06G 7/48; G06G 7/58; G06F 19/00; G01N 33/48; G01N 33/50

(52) U.S. Cl. 702/20; 703/11

(57) ABSTRACT

An artificial neural network system for analyzing sequence motif content for prediction of antisense oligonucleotide-target activity is disclosed. The system was developed for high specificity predictions, with cross-validation used to rigorously test against the database that was used in the development of the system. The system is able to choose effective oligonucleotides leading to >75% reduction in RNA target expression with >55% accuracy. This is in contrast to <10% success rate for trial-and-error oligonucleotide selection. Thus, the program provides a five-fold reduction in the number of oligonucleotides to be screened in vivo to find effective targets.

### CROSS-REFERENCE TO RELATED APPLICATIONS

This application claims the benefit of U.S. Provisional Application No. 60/262,993, filed Jan. 19, 2001.

### STATEMENT REGARDING FEDERALLY SPONSORED RESEARCH OR DEVELOPMENT

This invention was made with government support under NIH Genome training grant no. 5T32HG00042, NIH grant no. 2R01GM48152, and DOE grant no. DE-FG03-99ER62732. The government has certain rights in the invention.

### BACKGROUND OF THE INVENTION

This invention relates to antisense oligonucleotide technology. More particularly, this invention relates to an artificial neural network, method of use thereof, and method of making thereof for predicting active antisense oligonucleotides targeted to selected RNAs.

The development of reliable gene disruption strategies and their application in living cells is an important goal for cell and molecular biologists. Antisense oligodeoxynucleotide (ODN) technology allows the targeted down regulation of gene expression through the in vivo application of a short DNA molecule with reverse complementarity to a region on specific mRNA. The antisense molecule binds to the target RNA in the cell, causing RNase-H-dependent degradation by mechanisms that are still being studied. M. Y. Chiang et al., Antisense Oligonucleotides Inhibit Intercellular Adhesion Molecule 1 Expression by Two Distinct Mechanisms, 266 J. Biol. Chem. 18162-18171 (1991). This method has great utility in researching the role of genes in disease, and provides a powerful tool for understanding gene dynamics. It also shows promise for direct treatment of certain diseases such as AIDS and cancer through control of gene expression. E.g., T. Geiger et al., Antitumor Activity of a C-Raf Antisense Oligonucleotide in Combination with Standard Chemotherapeutic Agents Against Various Human Tumors Transplanted Subcutaneously into Nude Mice, 3 Clin. Cancer Res. 1179-1185 (1997); J. Jendis et al., Inhibition of Replication of Drug-resistant HIV Type 1 Isolates by Polypurine Tract-specific Oligodeoxynucleotide TFO A, 14 AIDS Res. Hum. Retroviruses 999-1005 (1998). Advances in chemistry have provided a basis to improve selectivity, stability, and specificity of action of ODN's, resulting in several antisense molecules reaching human clinical trials. E.g., A. M. Gewirtz, Myb Targeted Therapeutics for the Treatment of Human Malignancies, 18 Oncogene 3056-3062 (1999). However, in spite of some notable successes, a number of problems associated with the use of ODN's are not yet solved. A. D. Branch, A Good Antisense Molecule Is Hard To Find, 23 Trends Biochem. Sci. 45-50 (1998); C. A. Stein, Keeping the Biotechnology of Antisense in Context, 17 Nat. Biotechnol. 209 (1999).

When designing ODN's to target an RNA, there is a choice of many target sites, since the ODN is typically only about 20 nucleotides in length, as compared to a much larger RNA molecule. However, there is a great deal of variation in the efficacy of the ODN depending on the target site selected. E.g., C. F. Bennett et al., Inhibition of Endothelial Cell Adhesion Molecule Expression with Antisense Oligonucleotides, 152 J. Immunol. 3530-3540 (1994); S. P. Ho et al., Potent Antisense Oligonucleotides to the Human Multidrug Resistance-1 mRNA Are Rationally Selected by Mapping RNA-accessible Sites with Oligonucleotide Libraries, 24 Nucleic Acids Res. 1901-1907 (1996). Antisense efficacy is generally measured by applying an ODN and measuring the reduction in target RNA expression in vivo compared to one or more control experiments. When measured this way, the site-dependent variation of efficacy ranges from ODNs that completely knock out target RNA expression within the assay's limits to ODNs that appear to have no effect whatsoever on the target.

This presents a significant obstacle in the practical application of antisense technology. It is relatively expensive and time consuming to perform in vivo screening of multiple ODNs against a target to determine which is the most effective. Several in vitro approaches have been developed that reduce the time and cost factors, but these methods do not perfectly mimic the in vivo environment and thus have limited accuracy. S. P. Ho et al., supra; E. M. Southern et al., Discovering Antisense Reagents by Hybridization of RNA to Oligonucleotide Arrays, 209 Ciba Found. Symp. 38-44 (1997); O. Matveeva et al., Prediction of Antisense Oligonucleotide Efficacy by In Vitro Methods, 16 Nat. Biotechnol. 1374-1375 (1998).

Several computational approaches have been developed for predicting the efficacy of antisense ODNs. These methods utilize ODN and RNA sequence data to provide a ranking of target sites (and their complementary ODNs). Most of these methods are based on the hypothesis that ODN efficacy is determined by the affinity of the ODN for the target. In particular, structural and energetic considerations of ODN and mRNA are utilized to find those sites where ODN binding is favored. R. A. Stull et al., Predicting Antisense Oligonucleotide Inhibitory Efficacy: A Computational Approach Using Histograms and Thermodynamic Indices, 20 Nucleic Acids Res. 3501-3508 (1992); V. Patzel et al., A Theoretical Approach to Select Effective Antisense Oligodeoxyribonucleotides at High Statistical Probability, 27 Nucleic Acids Res. 4328-4334 (1999); S. P. Walton et al., Prediction of Antisense Oligonucleotide Binding Affinity to a Structured RNA Target, 65 Biotechnol. Bioeng. 1-9 (1999). It is difficult to assess the effectiveness of these methods or to use the results for comparative purposes. Each method used a different experimental data set for testing predictions. One work used only comparisons against in vitro binding assays. S. P. Walton et al., supra. The others were tested on limited data sets that were too small to demonstrate statistically significant generalization of the method to unseen data. Also, various performance metrics were used, making comparison between them difficult. Moreover, none of these methods was tested against a large database for providing meaningful statistics about the predictive properties of the system.

Though there is experimental support for structural and energetic mechanisms playing an important role in antisense efficacy, they are not necessarily the sole moderators. It was demonstrated that the single tetranucleotide motif TCCC, when present in an ODN, increases the likelihood of the ODN being effective from a background rate of less than 10% to about 50%. G. C. Tu et al., Tetranucleotide GGGA Motif in Primary RNA Transcripts. Novel Target Sites for Antisense Design, 273 J. Biol. Chem. 25125-25131 (1998). This observation is difficult to explain strictly from an accessability or energetics standpoint.

Artificial neural networks have been used or suggested for identifying protein-coding regions in DNA, G. D. Schellenberg et al., U.S. Pat. No. 5,449,604 (1995); E. C. Uberbacher & R. J. Mural, Locating Protein-coding Regions in Human DNA Sequences by a Multiple Sensor-Neural Network Approach, 88 Proc. Nat'l Acad. Sci. USA 11261-11265 (1991), and for identifying related amino acid sequences and nucleotide sequences and defining structural or functional domains in polypeptides, S. J. Korsmeyer, U.S. Pat. No. 5,622,852 (1997); S. J.

Korsmeyer, U.S. Pat. No. 5,700,638 (1997); S. J. Korsmeyer, U.S. Pat. No. 5,834,209 (1998); S. J. Korsmeyer, U.S. Pat. No. 5,856,171 (1999); S. J. Korsmeyer, U.S. Pat. No. 5 5,942,490 (1999); S. J. Korsmeyer, U.S. Pat. No. 5,955,595 (1999); R. C. Austin et al., U.S. Pat. No. 5,817,461 (1998); F. Bard et al., U.S. Pat. No. 5,811,514 (1998); G. R. Crabtree et al., U.S. Pat. No. 5,837,840 (1998); J. J. Harrington et al., U.S. Pat. No. 5,874,283 (1999); W. Funk, U.S. Pat. No. 6,025,194 (2000); M. J. Guimaraes et al., U.S. Pat. No. 5,858,707 (1999). None of these patents or publications discloses or suggests using neural networks for predicting target sites for antisense activity.

While methods for finding active antisense oligonucleotides are known and are generally suitable for their limited purposes, they possess certain inherent deficiencies that detract from their overall utility. For example, trial and error methods are labor-intensive, time consuming, inefficient, and expensive.

In view of the foregoing, it will be appreciated that providing neural networks for predicting active antisense oligonucleotides, methods of use thereof, and methods of making thereof would be significant advancements in the art.

### BRIEF SUMMARY OF THE INVENTION

An illustrative method according to the present invention for predicting antisense activity of an oligonucleotide for down-regulating expression of a selected RNA comprises:

(a) developing an artificial neural network embodied on a computer-readable medium comprising

(i) constructing a database comprising sequence data of oligonucleotides tested in vivo for activity in down-regulating expression of RNAs and activity data corresponding to said sequence data,

(ii) providing an input layer containing a selected number of input nodes, optionally at least one hidden layer comprising a plurality of hidden nodes having full connectivity to said input nodes, and an output layer comprising at least one output node connected to said plurality of hidden nodes, if present, or to said input nodes,

(iii) mapping sequence motifs of a preselected length found in the sequence data contained in the database, entering counts for each of said sequence motifs in selected input nodes of the input layer, and entering the activity data correlated with said counts of said sequence motifs, and

(iv) training the artificial neural network having the counts entered in the input layer thereof such that the artificial neural network produces an output in the output layer, wherein said output comprises a measure of predicted activity correlated with sequence motif counts for a test oligonucleotide; and

(b) mapping sequence motifs of the preselected length present in a nucleotide sequence of a test oligonucleotide complementary to at least a portion of said selected RNA, and entering counts of said sequence motifs present in the nucleotide sequence of said test oligonucleotide in the input layer of the artificial neural network; and

(c) obtaining output of the predicted antisense activity of the test oligonucleotide for down-regulating expression of said selected RNA.

An illustrative method according to the present invention for making an artificial neural network, embodied on a computer-readable medium, for predicting antisense activity of oligonucleotides for down-regulating expression of a selected RNA comprises:

(a) constructing a database comprising sequence data of oligonucleotides tested in vivo for activity in down-regulating expression of RNAs and activity data corresponding to said sequence data;

(b) constructing an artificial neural network comprising an input layer containing a selected number of input nodes, optionally at least one hidden layer comprising a plurality of hidden nodes having full connectivity to said input nodes, and an output layer comprising at least one output node connected to said plurality of hidden nodes, if present, or to said input nodes;

(c) mapping sequence motifs of a preselected length found in the sequence data contained in the database, entering counts for each of said sequence motifs in selected input nodes of the input layer, and entering the activity data correlated with said counts of said sequence motifs; and

(d) training the artificial neural network having the counts entered in the input layer thereof such that the artificial neural network produces an output in the output layer, wherein said output comprises a measure of predicted activity correlated with sequence motif counts for a test oligonucleotide.

An illustrative artificial neural network embodied on a computer-readable medium according to the present invention comprises:

(a) an input layer containing a selected number of input nodes;

(b) optionally at least one hidden layer comprising a plurality of hidden nodes having full connectivity to said input nodes; and

(c) an output layer comprising at least one output node connected to said plurality of hidden nodes, if present, or to said input nodes;

wherein sequence motifs of a preselected length found in a database comprising (i) sequence data of oligonucleotides tested in vivo for activity in down-regulating expression of RNAs and (ii) activity data corresponding to said sequence data are mapped and counts for each of said mapped sequence motifs are entered in selected input nodes of the input layer, and the activity data correlated with said counts of said sequence motifs are also entered in said selected input nodes of the input layer, and then the artificial neural network is trained such that the artificial neural network produces an output in the output layer, wherein said output comprises a measure of predicted activity correlated with sequence motif counts for a test oligonucleotide.

### BRIEF DESCRIPTION OF THE SEVERAL VIEWS OF THE DRAWINGS

FIG. 1 shows a graph of mean-squared-error (MSE) versus epoch for an illustrative network during back-propagation training, wherein the top curve represents the MSE for the untrained test cases (Test Set Error), and the bottom curve the MSE for the data used in training (Training Set Error); the error on the training set data decreases, while the error predicting the test set data increases, a classic case of over raining.

FIG. 2 shows a schematic diagram of an illustrative Chi-40 network, according to the present invention.

FIG. 3 shows a graph of the log scale output training function from equation 7, with c=100.

FIG. 4 shows Receiver Operating Characteristic (ROC) curves for an illustrative network reported herein comparing take-one-out (Minus One Oligo) cross validation to minus-one-RNA cross validation; also shown for reference is the single point representing the sensitivity and specificity of the method of G. C. Tu et al., supra, on this database.

FIG. 5 shows plots of ROC area versus training set size for an illustrative Chi-40 network using the original 372-oligonucleotide database described herein.

FIG. 6 shows a comparison of ROC curves for illustrative Chi-40 networks trained using two different activity transforms, plus an illustrative network trained using the actual activity data without transformation; for the log-transform data, the inverse transform is applied to the network output before ROC calculation.

FIG. 7 shows ROC curves for Gibbs free-energy based predictor, Chi-40 neural network predictor (take-one-out cross validation), and a logistic regression combining the two into a probability score.

FIG. 8 shows regression of neural-network-predicted versus actual ODN activities.

### DETAILED DESCRIPTION

Before the present artificial neural networks, methods of use thereof, and methods of making thereof for predicting active antisense oligonucleotides are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

Statistical links between short textual motifs (primarily 3-mers and 4-mers) and antisense ODN effectiveness have been explored. Using a database of 349 ODNs to be described below, it was found that there are several dozen motifs, aside from TCCC, correlated with in vivo antisense action. O. V. Matveeva et al., Identification of Sequence Motifs in Oligonucleotides Whose Presence Is Correlated with Antisense Activity, 28 Nucleic Acids Res. 2862-2865 (2000). The presently described invention relates to a way to use these observations as part of a predictive tool for antisense efficacy.

Antisense oligodeoxynucleotides can vary in length. Short ODNs lack specificity, and long ODNs can be difficult to produce, target, and deliver. In standard practice ODNs of about nucleotide residues (nt) in length are used, because they usually strike an optimal balance of these factors. ODNs substantially shorter than 20 nucleotide residues can be used provided that sufficient specificity is obtained, and ODNs substantially longer than 20 nucleotide residues can be used provided that such ODNs can be adequately synthesized, targeted, and delivered. Therefore, the only limit on the length of ODNs is functionality. ODN sequences present in the database range from 10 to 22 nt in length, with most of them about 18-22 nt in length. For the purpose of clarity, the remainder of the discussion will focus on ODNs and their sequences, not the complementary RNA targets. As used herein, the term “complementary” refers to nucleic acid strands that are antiparallel and wherein A and T (or U) residues bind to each other and G and C residues bind to each other.

For an ODN sequence of length n that is decomposed into motifs of length l, there are (n−l+1) motifs contained in the ODN sequence. With the four-letter DNA alphabet (A, C, G and T), there are 4^{l} possible motifs of length l. For example, there are 256 possible tetranucleotide (4-mer) motifs. If all possible motifs at a given length are enumerated in some fashion (e.g., alphabetical order), then an ODN sequence can be represented as a set of numbers of the counts for each possible motif in that ODN sequence. Motifs are analyzed in a position-independent manner. Since a 20-nt ODN sequence is composed of 17 overlapping 4-mers, most of the motif counts will be zero, with a few l's and an occasionally higher number for multiple occurrences of a motif. This representation is not a unique mapping from ODN sequence to motif counts, since there can be more than one way a set of motifs can be scrambled into different ODN sequences. However, observations have not indicated any positional dependence for the statistically significant motifs within ODN sequences in efficacy determination, so spatial ordering may not be necessary, and has the advantage of representational simplicity.

Given the above mapping of ODN sequences to 1-mer motif sets, the task of a predictive method is to find and generalize for correlations between the motif set and the efficacy of the ODN. Typically, efficacy is represented as the percentage of control (e.g., scrambled ODN) at which the target RNA is expressed after ODN application, so activities lie in the [0.0,1.0] interval with lower activities being better (more expression reduction). This mapping can be represented as

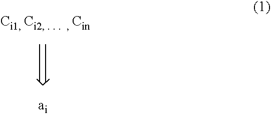

where the cᵢⱼ represent the counts for the alphabetically ordered motifs within the ODN sequence, n=4^{l}, and aᵢ is the assayed RNA activity for ODN i.

The present system uses feed-forward artificial neural networks (ANNs) to predict efficacy based on the mapping in equation (1). Artificial neural networks have been used for a number of biological sequence-analysis tasks with success. C. H. Wu, Artificial Neural Networks for Molecular Sequence Analysis, 21 Comput. Chem. 237-256 (1997). They allow the formation of an arbitrary mapping between two data sets containing statistical correlations through the use of a training process.

Several means of cross validation were applied to measure the generalization ability of the neural networks. In general, the present method can select ODNs likely to be active with approximately a 55% success rate. The method is surprisingly accurate given that it foregoes any consideration of binding site accessibility or energetics. The methods used to achieve these results, including the cross validation, network architectures, and training methods used are discussed below.

### Methods

Performance measurement. Many experiments were performed to explore the properties of the various parameters available. However, universal to all such explorations is performance measurement. There are a number of approaches available for measuring the accuracy and performance of a prediction method. There are tradeoffs with many of the approaches. Common and easy to understand measures of performance are given by specificity (Sp) and sensitivity (Se),

$$Se = \frac{Tp}{Tp + Fn} and Sp = \frac{Tn}{Tn + Fp} , \qquad ( 2 )$$

where Tn is true negative predictions, Fn is false negative predictions, Tp is true positive predictions, and Fp is false positive predictions. Another related measure is the probability of a positive prediction being correct, given by

$${P}^{+} = \frac{Tp}{Tp + Fp} . \qquad ( 3 )$$

One problem with these measures is that they rely on the use of a specific threshold that distinguishes between positive and negative cases in the data. Sampling at only one threshold value gives a very limited perspective on the performance, since across the space of possible thresholds there is natural variation due to noise.

A standard approach for dealing with this problem is called Receiver Operating Characteristic (ROC) analysis. It comprises sampling the values of Sp and Se at many different thresholds spanning the range from minimum to maximum model output (prediction values). J. A. Hanley & B. J. McNeil, The Meaning and Use of the Area under a Receiver Operating Characteristic (ROC) Curve, 413 Radiology 29-36 (1982). For continuous models, there is generally an inverse relationship between specificity and sensitivity. For example, a random number generator produces an ROC curve that approximates the diagonal, with an average area under the curve of 0.5. The perfect model would exhibit no tradeoff between specificity and sensitivity and thus would have an area of 1.0. Thus, two important aspects of an ROC curve are the area contained and the way in which this area is distributed.

For the present task, ROC curves are sought that have their area distribution biased towards the high specificity end of the curve. The goal of this work is to find a few ODNs that have a high likelihood of success for a given RNA. It is not a problem if there are many false negatives (low sensitivity) as long as enough targets are found that they are likely to be active in vivo. Although reporting the area under an ROC curve is a concise means of overall performance measurement, it does not fully indicate how a model will work on the present problem. ROC areas are reported herein, but are qualified against a discussion of the shape of the distribution.

It should also be noted that the measurement of ROC curves is still dependent on a set threshold for the real activity values of the ODNs. The ROC method requires that the data be classified in positive or negative categories for comparison against results at various threshold values for the predicted value. The choice of this threshold for the in vivo data has some effect on the measured ROC values for various models. It is clear that this threshold must be chosen so that the set of negatives or positives is not too small. For experiments herein, a value of 0.25 of the control value was used.

Another approach used for reporting antisense prediction accuracy is the correlation coefficient R and the significance (P) value. R. A. Stull et al., supra; S. P. Walton et al., supra. This measure is free of threshold dependencies, and provides a good indicator of whether predictions relate well to experimental measurements. However, it has the problem that it is difficult to translate R-value measures into a meaningful metric of direct accuracy (e.g., the probability of a correct prediction). For this reason the use of this measure is not emphasized herein.

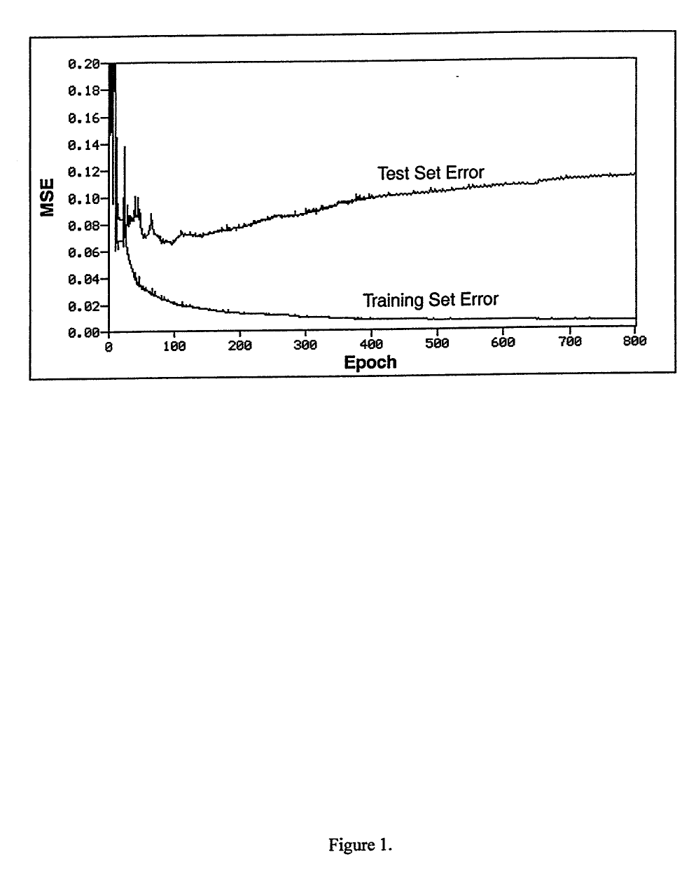

Cross-validation. Several cross-validation approaches were used for assessing generalization. The critical property sought in cross validation is that with training on one data set, the model is able to extract, or induce, general observations that will lead to useful predictions for data that have not been seen previously. The first approach used was the “minus 10%” system, where 10% of the database was randomly selected as the “test set.” Training was performed using the remaining 90%, and after training, performance tested on the unseen 10%. This method was used for early manual experiments to determine the overall range of neural network architectures and learning parameters worth testing further. The Stuttgart Neural Network Simulator (SNNS), used for all experiments, provided the capability to monitor generalization during training. A graph was produced measuring the sum-of-squared errors (SSE) between expected output and actual output for each example, with error plotted versus the number of training cycles (epochs). A comparison of the SSE for the training versus test set indicated how well the model was generalizing. For example, FIG. 1 shows a graph of mean-squared-error (MSE) versus epoch for an illustrative network during back-propagation training, wherein the top curve represents the MSE for the untrained test cases, and the bottom curve the MSE for the data used in training. The error on the training set data decreased, while the error predicting the test set data increased, a classic case of over-training. Thus, it was clear from early experiments that over-training was an issue to be contended with.

To more rigorously test promising parameter sets, a “take-one-out” approach was used. Using PERL scripts according to procedures well known in the art, a process was automated whereby single ODN sequences were sequentially selected from the database as test cases. The remainder of the database in each case was used as the training set. The model was trained with the training set, then tested for accuracy in predicting the single test ODN sequence. The result was recorded for each test ODN sequence, and the procedure was repeated for each ODN sequence in the database. On a modern desktop machine with typical training parameters (500-1000 training cycles or epochs), this process takes 3-6 hours. This approach is also referred to as “minus-one-oligo” or “-oligo” cross-validation.

There are several ODN sequences in the database that have significant sequence overlap. This is due to experiments where researchers tested ODNs by walking along an RNA target in increments of 2 nucleotides. There is also one experiment incorporated into the database where the same region of an RNA was tested using three different length ODNs. And finally, there is one ODN present that was tested by two different laboratories. Take-one-out cross validation may not accurately reflect generalization in this case since the train and test data are not completely independent. So, a new regime was developed to ameliorate this concern, called “minus-one-RNA” (abbreviated “-RNA”). This system comprises first removing all ODN sequences derived from a single reference for a given RNA as a test set, and then using the remainder of the database as the training set. This is repeated for each RNA. However, there are a few RNAs that were tested by more than one reference, such as human endothelial leukocyte adhesion molecule I. C. F. Bennett et al., supra; C. H. Lee et al., Antisense Gene Suppression against Human ICAM-1, ELAM-1, and VCAM-1 in Cultured Human Umbilical Vein Endothelial Cells, 4 Shock 1-10 (1995). Therefore, this cross validation was made more rigorous by excluding all examples for a given RNA name as test cases, regardless of source.

In standard feed-forward neural networks using back-propagation training, the network is first initialized with random connection weights. This randomly-chosen starting point can have a significant impact on how well a particular model generalizes for the problem. Because of this, for all experiments testing a given set of network parameters, more than one network was tested at a time (typically five), with the only difference between each network being the randomly initialized starting weights.

Database. The database used for this work comprised a set of ODN sequences collected from the literature. C. F. Bennett et al., Inhibition of Endothelial Cell Adhesion Molecule Expression with Antisense Oligonucleotides, 152 J. Immunol. 3530-3540 (1994); C. H. Lee et al., Antisense Gene Suppression against Human ICAM-1, ELAM-1, and VCAM-1 in Cultured Human Umbilical Vein Endothelial Cells, 4 Shock 1-10 (1995); L. Miraglia et al., Inhibition of Interleukin-1 Type I Receptor Expression in Human Cell-lines by an Antisense Phosphorothioate Oligodeoxynucleotide, 18 Int'l J. Immunopharmacol. 227-240(1996); N. M. Dean et al., Inhibition of Protein Kinase C-alpha Expression in Human A549 Cells by Antisense Oligonucleotides Inhibits Induction of Intercellular Adhesion Molecule 1 (ICAM-1) mRNA by Phorbol Esters, 269 J. Biol. Chem. 16416-16424 (1994); J. L. Duff et al., Mitogen-activated Protein (MAP) Kinase is Regulated by the MAP Kinase Phosphatase (MKP-1) in Vascular Smooth Muscle Cells. Effect of Actinomycin D and Antisense Oligonucleotides, 270 J. Biol. Chem. 7161-7166 (1995); S. P. Ho et al., Potent Antisense Oligonucleotides to the Human Multidrug Resistance-1 mRNA Are Rationally Selected by Mapping RNA-accessible Sites with Oligonucleotide Libraries, 24 Nucleic Acids Res. 1901-1907 (1996); S. P. Ho et al., Mapping of RNA Accessible Sites for Antisense experiments with Oligonucleotide Libraries, 16 Nat. Biotechnol. 59-63 (1998); S. M. Stepkowski et al., Blocking of Heart Allograft Rejection by Intercellular Adhesion Molecule-1 Antisense Oligonucleotides Alone or in Combination with Other Immunosuppressive Modalities, 153 J. Immunol. 5336-5346 (1994); M. Y. Chiang et al., Antisense Oligonucleotides Inhibit Intercellular Adhesion Molecule 1 Expression by Two Distinct Mechanisms, 266 J. Biol. Chem. 18162-18171 (1991); B. P. Monia et al., Antitumor Activity of a Phosphorothioate antisense Oligodeoxynucleotide Targeted against C-raf Kinase, 2 Nat. Med. 668-675 (1996); C. L. D'Hellencourt et al., Differential Regulation of TNF alpha, IL-1 beta, IL-6, IL-8, TNF beta, and IL-10 by Pentoxifylline, 18 Int'l J. Immunopharmacol. 739-748 (1996); G. C. Tu et al., Tetranucleotide GGGA Motif in Primary RNA Transcripts. Novel Target Site for Antisense Design, 273 J. Biol. Chem. 25125-25131 (1998); A. J. Stewart et al., Reduction of Expression of the Multidrug Resistance Protein (MRP) in Human Tumor Cells by Antisense Phosphorothioate Oligonucleotides, 51 Biochem. Pharmacol. 461-469 (1996). The criteria for inclusion in the database were that at least 10 ODNs were tested and reported in the article, and at least one mismatch or scrambled ODN control was used in the reported results. The database currently has 349 ODN sequence entries that were screened to meet these rigorous criteria. This database is described more thoroughly in M. C. Giddings et al., A Web Database for Antisense Oligonucleotide Effectiveness Studies, 16 Bioinformatics 843-844 (2000). Some of the early experiments reported herein were performed on a larger database of 372 ODN sequences, which was later culled to 349 ODN sequences through establishment of stricter criteria and concerns about the quality of two specific references. The cross-validated performance of the methods reported here was not significantly impacted by the change.

Parameters of neural networks. There were many parameters to explore in constructing a neural network system for this problem domain. The main issues explored included motif length, network architectures, training methods, learning parameters, and input-output representation. Only a few of the most successful parameter combinations and their results are described here. However, a plurality of systems constructed according to the present invention performed well on the problem in cross-validation, so it is unlikely that the performance observed is an accident of one particular parameter set.

Network architecture. For all neural network experiments, the Stuttgart Neural Network Simulator (SNNS) was used. A. Zell et al., Recent Developments of the SNNS Neural Network Simulator, Aerospace Sensing Int'l Symp. 708-719 (Orlando, Fla., SPIE 1991) (http://www-ra.informatik.uni-tuebingen.de/SNNS/). The system comprises a kernel, batch language, and graphical interface. Initial experiments were usually carried out with the graphical interface followed by more thorough cross-validation testing utilizing the kernel, batch language, and custom PERL scripts.

For all experiments, standard feed-forward networks were used. D. E. Rumelhart & J. L. McClelland, Parallel Distributed Processing (MIT Press 1986); P. De Wilde, Neural Network Models (Springer-Verlag 1997). Initial work explored networks comprising 2 and 3 layers where the input field comprised one node per motif. With tetranucleotide motifs, this implies 256 input nodes. The hidden layers, which are layers of nodes having no direct connection to the outside world (only to other nodes), ranged from 16 to 4 nodes. The output layer always comprised one node, trained to correspond to ODN activity mapped through various functions described below.

Various supervised learning algorithms provided by SNNS were tested on the problem, but the majority of experiments were performed using the back-propagation (backprop) algorithm with a momentum term. D. E. Rumelhart et al., Learning Internal Representations by Error Propagation, 1 Parallel Distributed Processing: Foundations 318-364 (MIT Press 1986). The back-propagation method performs connection weight adjustments to minimize the difference between the training signal and the actual network output at the output nodes. It is a gradient-descent method that recursively adjusts weights to reduce the error of the network's output for a given input pattern. The rate of descent is controlled by the learning parameter η. In the back-propagation momentum method, the learning equation utilizes two additional parameters, μ and c, to reduce oscillation during learning and avoid flat spots in the error space. Experiments indicated that the back-propagation momentum method generalized better than the basic back-propagation method.

During training and testing, a network is executed once per ODN sequence, with input node values set according to the count of the various motifs present (or not) within the ODN sequence being analyzed. The output node training signal is a function of the measured activity for the ODN. The functions used for scaling and thresholding input and output are discussed further below.

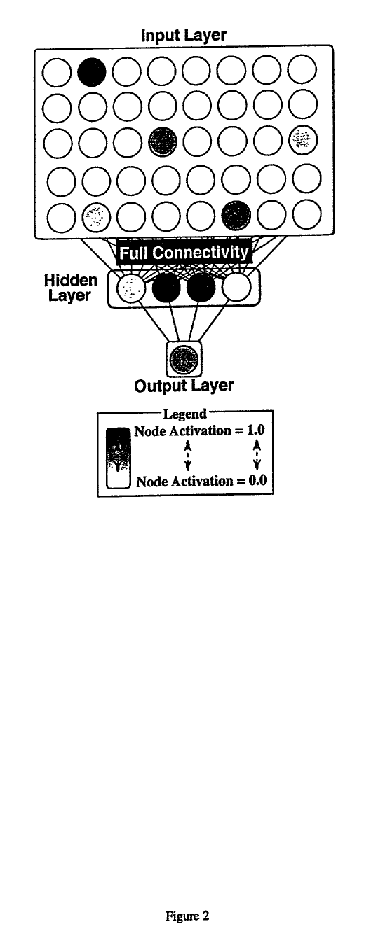

Parameters were explored to determine a range that avoids over-training while providing sufficient training for the model to generalize. In general, the networks analyzing tetranucleotide motifs worked best with low η or a low number of epochs. Some experiments with fully-connected networks analyzing all 256 tetranucleotide motifs had problems with over-learning of the training set. To address this and provide a more computationally efficient network, a new architecture was developed. Rather than utilizing all 256 4-mers, only those motifs exhibiting a statistical correlation in their presence to ODN activity were used. Specifically, a χ² test for significance was performed on the motifs for all ODN sequences in the database, G. R. Norman & D. L. Streiner, PDQ Statistics (Mosby, St. Louis 1997), and they were ranked from most to least significant. An advantageous model uses the top 40 4-mers, which are mapped to 40 input nodes of a three-layer network (with 4 hidden nodes). This architecture is represented in FIG. 2, and is dubbed the Chi-40 network. Other similar architectures can be used advantageously according to the principles of the present invention. For example, selecting the top 50 4-mers would produce a Chi-50 network, and so forth. The minimum number of 4-mers that can be selected according to this scheme is limited only by functionality. The maximum number of 4-mers that can be selected such that there is one node per sequence motif is 256, but greater efficiency is obtained by reducing this number.

In addition, it was discovered that a linear activation function used on the output neuron aids performance (all other nodes retain logistic activation functions). This result held true among a variety of training conditions. The reason for this is not clear. Using a linear activation function on the output node has the side-effect that the network can produce values that are not constrained within any particular range, so, for example, it may output negative values as predictions. To address this, the output prediction values can be normalized, for example, with a linear function that rescales the outputs to lie on the range [0, 1].

Motif lengths. Statistical analyses show correlations between 3-mer and 4-mer motif content and the activity of an ODN, but the question of the size of the motifs at which the correlation is maximized remains open. There is probably a motif length l at which this is optimal. Unfortunately, the data set used herein is not large enough to confirm this definitively. Consider again the relation between motif length and the number of possible motifs, 4^{l}. For 5-mers, the number of motifs grows to 1,024. For a data set of 349 ODN sequences with an average of 17 motifs apiece, the statistics of 5-mers are such that a few motifs may not be present at all, some are present only once or twice, and even the most common ones appear only 5-10 times. This makes meaningful statistical analysis difficult, and the problem is exacerbated greatly with increases in motif length. Given the database size, the largest motif size presently practical for meaningful analysis is 4.

Some work was done both statistically and with neural networks on composition bias (l=1). It became clear that there was some compositional bias present (favoring C), but predictions based on this were relatively weak and nonspecific. Some exploration was also performed with di-nucleotide and tri-nucleotide motifs, and based on these limited tests, it appears that with each step up to the tri-nucleotide level, prediction accuracy (and generalization) for the neural networks improves. The transition from l=3 to l=4 is not so straightforward. At this transition, an issue emerges affecting generalization. At l=4 with 256 motifs, it becomes possible for a neural network to learn to distinguish each individual ODN sequence in the training set by its input pattern. This leads to the condition illustrated in FIG. 1, where performance during training (SSE on the training set) improves to the point at which there is very little error, whereas performance on the cross-validation test cases worsens as the network over-learns. The Chi-40 network addresses this issue for the 4-mer analyses. Unfortunately, the optimal motif length question cannot be answered, except to say that up to a length of four nucleotides predictions improve.

Input/output representation. The way data are mapped for input to the network and output from the network has a substantial impact on performance. The data need to be transformed so they can be represented by node activation values in the network. Fundamentally, the input for the problem is a sequence string from the alphabet (A, C, G, T), and the output is a number, relating to the activity of that string in vivo. There are several issues to be considered in attempting to model this mapping from string to number. The most fundamental is whether or not the string itself contains enough information to determine the activity value. This in turn depends on the mechanisms of antisense action, which are not fully elucidated. Both the present work and the work of G. C. Tu et al., supra, indicate that certain short sequence motifs contained in the string have a statistical correlation with activity. But this by no means implies that motifs are the sole determinant of efficacy. In fact, a strongly-held theory is that the primary determinant of ODN efficacy is the thermodynamics of ODN-target binding. In this case, it is believed that target RNA structure plays a role, and thus the string representing the ODN sequence alone does not contain adequate information. It is believed that there are likely to be at least two mechanisms playing a role in antisense efficacy, one of those being motif content. It is not expected that this model using motifs will achieve perfect accuracy in isolation, but achieves high enough accuracy to be useful on its own and even more useful in combination with other approaches. Given this view and the results presented herein, there appears to be enough information within the ODN sequence alone to produce a useful computer model of antisense activity.

A second issue of input representation is how to map the information contained in the string onto the network. A simple choice would be to utilize one node per character position in the ODN sequence. This has not been tested, but it seems unlikely to work well. It would be difficult for a network to map a string of arbitrary position in the input field to a decomposition of positionally-independent motif counts. Instead, the approach used herein was to perform decomposition into positionally-independent motifs before presenting the data to the network. It is straightforward to design a network to find correlations between such a decomposed input set and ODN activity (assuming such correlations exist in the data). Specifically, as described in equation 1, the input comprises motif counts for a given ODN. These can be scaled from whole numbers to a smaller real-valued range by the equation:

$$a = \frac{10 c}{l + 1 - n} , \qquad ( 5 )$$

where c is the number of times the motif of length n occurs in the ODN of length l. The constant 10 is used to scale these numbers so that they lie approximately on the range 0-1. This makes debugging the input and output simpler. This equation was chosen so that the proportion of the ODN comprised of the motif corresponding with that node is represented, rather than the direct count. This helps normalize for different-length ODNs in the input, and appears to improve performance of the predictions.

Training methods. The system has a single output unit, corresponding to the activity of the ODN. There are choices available about whether the output node is trained directly with the continuous-valued activities measured in a lab, or a secondary function thereof. Experiments training the output node directly with measured activity were not the best at generalizing. So other training functions were tested. Both a binary threshold function with a cutoff of 0.25 and a 3-way threshold were tested. The 3-way threshold is given by:

$$o = \{ \begin{matrix}0 , & act \leq 0.25 \\ 0.25 , & 0.25 < act \leq 0.5 \\ 1.0 , & act > 0.5\end{matrix} \} . \qquad ( 6 )$$

Training with threshold functions applied worked better than direct activity data training. Subsequent to this observation, it was noted that measurements at the low end of the RNA activity scale are generally more experimentally reproducible. Since the ODNs were tested at different labs using various concentrations, those with only slight effects are more susceptible to measuremental variation. For example, an ODN that reduces RNA expression to 0% (within measurement limits) of the control at 100 μM applied concentration will likely produce a very similar reduction at 50 μM, say to 0.5%. In contrast, an ODN that reduces RNA expression to 70% at 100 μM may exhibit much less remarkable effect at 50 μM (with a simplistic approximation of kinetics the expression level might be 85%).

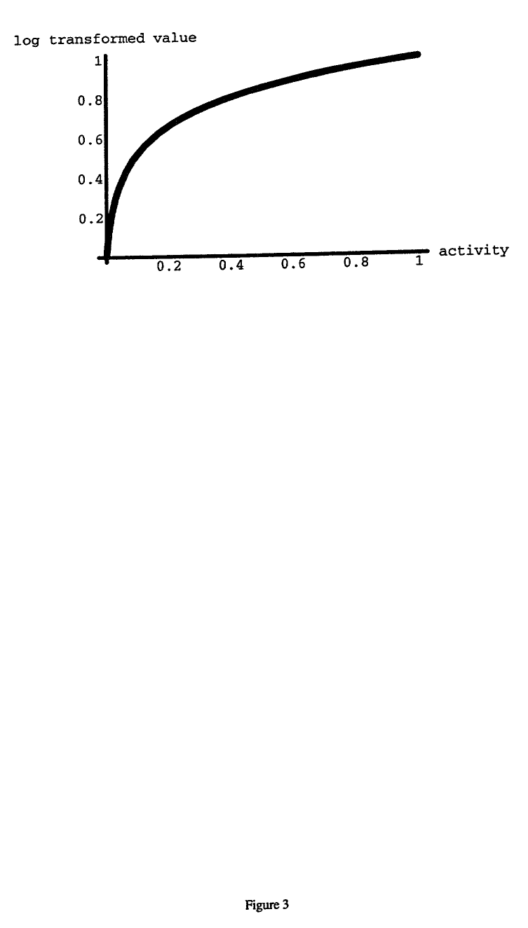

Given these observations, a log-scale transform function was developed to emphasize the differences amongst high-activity ODNs while de-emphasizing the differences between low-activity ODNs, by essentially grouping the latter in a very narrow region. The function is:

$$o = \frac{log ( 1 + act xc )}{ln ( 1 + c )} \qquad ( 7 )$$

where c is a scale constant for which the value of 100 was used. This value was determined by a few trial-and-error experiments. This function has the form shown in FIG. 3. Since an illustrative output node of the network uses a linear activation function, the use of eq. 7 is partially equivalent-to defining a new activation function for the node. This may explain why use of a linear activation function works better than a logistic function on the output node. The logistic function has a form that tends to emphasize differences in the central region of its curve, which is not ideal for the present task.

Combination approaches. Several approaches were tested wherein the predictions of multiple networks were combined or the predictions of network(s) with other methods were combined. The simplest approach, which is in essence a “voting” scheme, averages the outputs of several selected networks when a single “ODN sequence” is presented to each of them. Another approach to combining several predictors is logistic regression. D. W. Hosmer, Applied Logistic Regression (Wiley 1989); http://m2.aol.com/johnp71/logistic.html. This is a process where a logistic transform equation is used in combination with a linear regression of the transformed data to provide a probability estimator based on a set of independent variables. In fact, this process can be used directly for activity prediction with the motif counts as the independent variables. Matveeva et al., supra, explored this possibility. The downside of this approach is the difficulty of analytically maximizing the likelihood estimator over such a large set of independent variables (all motifs, or a large portion thereof). The algorithms tested demonstrated some instability, particularly for those motifs for which there are few or no examples.

For the present work, the regression is applied for a much simpler task: combining the outputs of a few predictors into one overall probability score of an ODN being active. This was used to combine the predictions of several networks, as well as combining a neural-network prediction with an estimator of the free-energy change associated with ODN-RNA duplex creation. This was calculated using the dinucleotide energies given by N. Sugimoto et al., Thermodynamic Parameters to Predict Stability of RNA/DNA Hybrid Duplexes, 34 Biochemistry 11211-11216 (1995).

Logistic regression can also be used for another purpose. Interpreting the output (prediction) of a neural network without some kind of normalization, especially those with linear activation functions, can be difficult. Logistic regression can be used to map the outputs of a network into the more useable form of probability values. The process comprises first performing cross-validation on the network using the minus-one-RNA or take-one-out method, and then using the activity prediction results as the independent variable to calibrate the regression coefficient. This provides a function that maps from the network output to an estimator of the probability of a given ODN being active.

### Results and Discussion

Many experiments were performed testing various network architectures, training parameters, motif lengths, and learning algorithms. A problem that can arise with this type of parameter space exploration is that, given enough trials, one will eventually find by chance a combination of parameters that will work well in cross-validation on the particular data set studied, but will not generalize to other data. However, in this work there was a multiplicity of parameter sets that produced working neural network predictors for the problem, with only relatively small variations in performance among them. It is quite unlikely that selection of multiple working predictors would occur by chance under the conditions used herein, unless there is something peculiar about the database. The second counter addresses the database issue. Experimental results verified that the motif statistics observed for the present database are valid for a separate and larger database. O. V. Matveeva et al., supra. This provides substantial confidence that the neural network generalizations are not due to some pathological feature of the database, but are in fact genuine.

It is possible to question whether the performance of a specific network chosen by good results in cross validation might be artificially high due to this phenomenon. Without a larger database, this is difficult to test. However, performance across a large set of experiments may be a useful indicator. An experiment was done where 400 networks were tested using minus-one-RNA cross validation, where the only difference among networks was the random initialization of their weights. The ROC curves produced ranged from 0.55 to 0.76 in total area. Though this is a wide spread of values, it is notable that of 400 experiments, all produced ROC curves with areas greater than random predictions would have yielded. The averages over this large set of networks also provides some information. The average ROC curve area was 0.65. With a threshold of −0.05, the averages of other measures were: P⁺=0.46, Tp=10.7, Fp=12.6, Sp=0.96, and Se=0.183. Therefore, the average network in this experiment will predict well enough to be useful in locating effective ODNs. Also, there are 57 networks in this experiment producing ROC curve areas greater than 0.7. It is believed this is a good indicator that a well-performing network can be chosen from the set and used without significant concern that its performance is by chance.

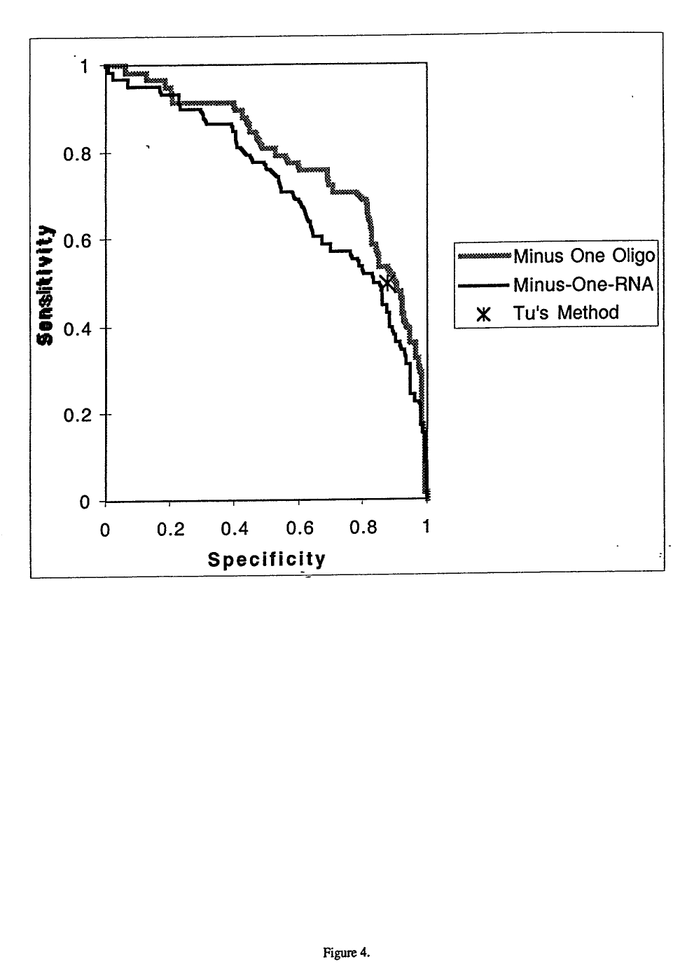

As mentioned above, originally take-one-out cross validation was used, then the concern arose that there could be some information “leakage” from the training set into the test set. This was tested by using the same five randomly-initialized Chi-40 networks in two different experiments with exactly identical parameters. The only difference is that one experiment used take-one-out cross validation and the second experiment used minus-one-RNA cross validation. The average ROC area for the 5 networks using the take-one-out cross validation was 0.69, and for minus-one-RNA cross validation was 0.65. The ROC curves for network number 5 are illustrated in FIG. 4. Though overall ROC area dropped, in the high-specificity region, prediction ability was not significantly altered. This supports the hypothesis that highly effective ODNs are the most consistent experimentally, and are also the most predictable based on motif content.

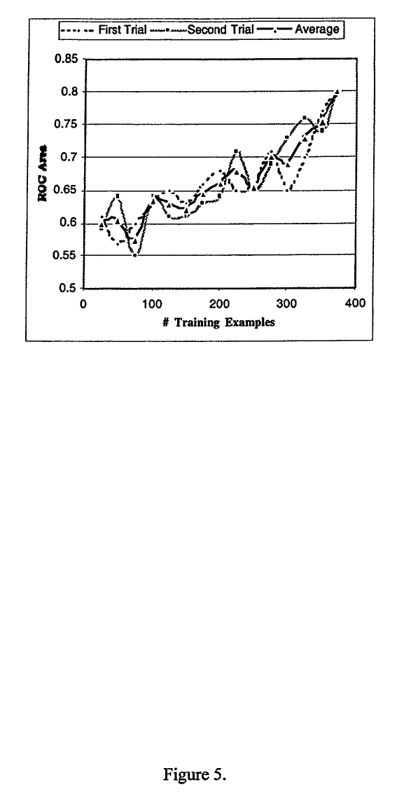

The reduction in overall accuracy due to the switch from take-one-out to minus-one-RNA cross validation has two readily apparent explanations: (a) that the elimination of some redundant ODN sequences eliminates information leakage that was artificially inflating the performance measures; or (b) that the reduction in training set size available with minus-one-RNA cross validation is impacting accuracy. To understand the impact of the latter, an experiment was performed measuring the relation between training set size and prediction accuracy. This was done in a manner similar to the take-one-out cross-validation, except that the training set size was varied from 25 ODN sequences to the full database. Each ODN sequence in the database was used as a test ODN sequence for one trial, and the training set was selected randomly from the remaining database. FIG. 5 shows the results of two such experiments. The graph shows a clear dependence of prediction accuracy on the size of the training set. The bumpiness of the curves is due to the random selections of training sub-sets. It also appears that at the terminus of the experiment, using the full data set minus the test ODN sequence, the slope is still upward.

This experiment provided two pieces of information. One is that the accuracy limit of the analysis method has not been reached using the present data set. It is likely that more data would improve the predictions further, though it is not possible to predict by how much. It also may explain the drop in accuracy observed using minus-one-RNA versus take-one-out cross validation. The average test set removed from the database in minus-one-RNA cross validation comprised 28 ODN sequences. Looking at the plot in FIG. 5, this translates into a reduction of almost 0.05 in ROC area due to the loss of this many training examples when compared to the take-one-out method. So, once this is factored out, it appears that there is not a substantial difference in accuracy of prediction when tested by minus-one-RNA versus take-one-out cross validation.

The selection of the χ²-ranked tetranucleotide motifs is performed once for the whole data set. Theoretically, this might be done in a cross-validated fashion for each selection of training and test sets. In practice, this does not seem necessary, since the statistics generally change very little with the removal of 0.3% of the data (1 ODN sequence). It has little impact on the top-40 set chosen as the input field.

Various network architectures were tested. Original experiments used variations on 2-, 3-, and 4-layer (2 hidden) feed-forward networks with an input field representing all tetranucleotide motifs. These networks achieved some successes in generalization, but were discovered to be particularly sensitive to over-learning as illustrated by the error signal shown in FIG. 1. The best network in these experiments achieved an ROC curve area of 0.78, however, more typical was ROC areas in the 0.60-0.70 range. It is believed the problematic generalization in these architectures is because the total number of nodes and connections is large enough that the network can memorize every ODN sequence pattern from the database. It is possible to adjust training parameters, minimizing learning rate and the number of training epochs to improve performance. However, early on it was discovered that the Chi-40 style network was easier to train without over-learning, so most experiments were subsequently performed with these networks. The Chi-40 style network limits the number of nodes and connections so that individual pattern learning becomes more difficult, thus enhancing generalization.

The experiments with various threshold and transformation functions on the data had clear effects. Original experiments used the activity data themselves, but it was soon discovered that the use of a threshold on these data produced better results. Various functions were tested, but the two most consistently useful were those given by equations 6 and 7. Both of these functions had the effect of transforming the data so that the less effective ODNs were grouped together, making them essentially indistinguishable from one another. Instead of the learning function attempting to precisely match the patterns of experimental noise present, it is focused upon the more general problem of separating the “good” from the “bad.”

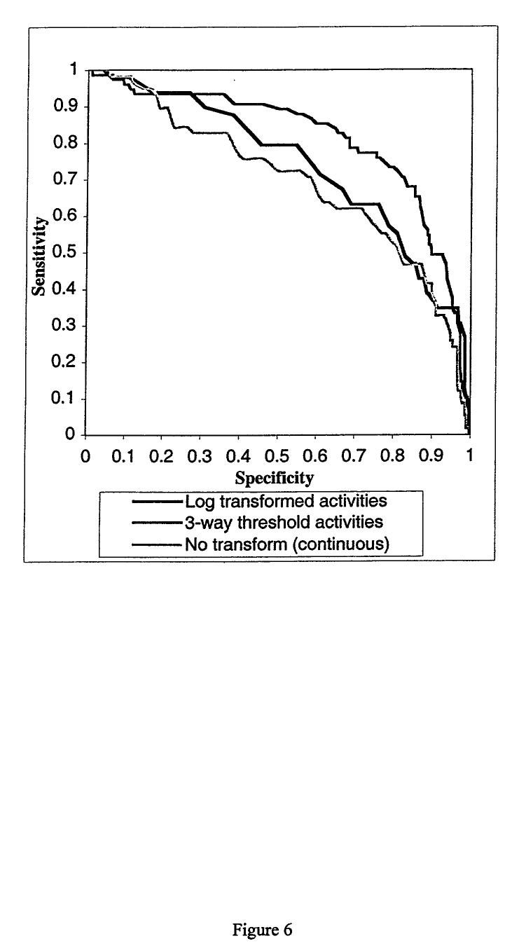

FIG. 6 illustrates the effects of the various functions on ROC curve performance. It is clear that the threshold function of equation 6 produces the highest overall ROC area. However, in the important high-specificity region, quite often the log function of equation 7 performs better. The network trained on non-transformed data is still doing a reasonable job of prediction, but in the high specificity region suffers somewhat. A possible explanation for the reasons behind the (slightly) better performance of equation 7 in the high-specificity region is that the differences in activity measured between active ODNs may be repeatable effects of motif content upon activity. If that is the case, equation 7 would work better because it not only retains, but enhances, the differences between the high activity ODNs.

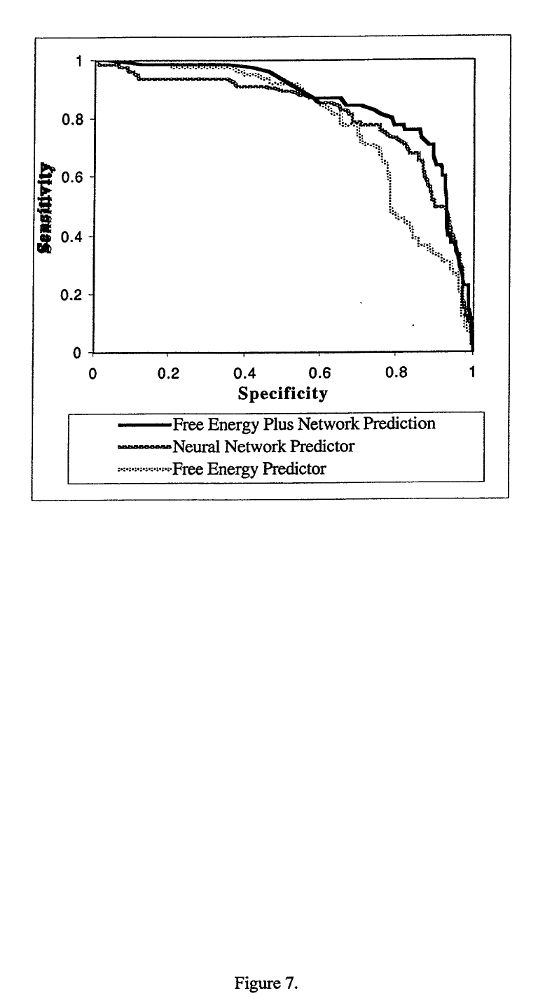

Another illustrative embodiment of the present invention was provided by experiments that examined combinations of several networks and other predictors into a single prediction. The voting experiments (where predictions of several networks were averaged) produced mixed results. Typically the voting produced results with ROC areas superior to that of the average network in the collective doing the voting. However, in many cases one or more of the individual networks within the collective produced more accurate predictions than the voting did. It may require a larger data set to determine whether these outperforming networks were an accident of the particular data used or not. Stronger results were provided by combining a neural network score with a straightforward AG calculation for binding between ODN and target. These were combined using logistic regression into a single probability ranking for an ODN being active. The ROC curves are shown in FIG. 7. Surprisingly, the simple AG calculation performed well on its own. But in the high-specificity region, it did poorly. The combined prediction appeared to benefit by the strengths of both independent predictors yet suffer none of their weaknesses. The ROC area of the combined prediction was >0.8, one of the best results obtained.

It is important to put into perspective what all of these results might mean to someone who wants to apply the present invention for finding effective ODNs. This can done using the example of a specific network system, such as the network whose cross-validation results are shown in FIG. 4. It is a Chi-40 network, trained for 1000 cycles, with parameters μ=0.1, α=0.05, c=0.1 and training examples presented in a random order for each cycle. With take-one-out cross validation, the ROC area is 0.78. At a threshold of 0.10, there were 5 false positive predictions and 12 true positive predictions, for a P⁺ of 0.71. For comparison, using the same database Tu's method (G. C. Tu et al., supra) selected 29 true positive ODNs and 36 false positive ODNs, a P⁺ of 0.45. This reveals one problem with Tu's method, namely, it does not rank ODNs or provide a means of adjusting a threshold distinguishing between positive and negative predictions (which is why there is no ROC curve).

The reported results are based on predictions from the database in cross validation. However, the database contains the bias that there are more positive examples than are expected in the general population. Estimates vary for the frequency of finding active ODNs by random selection on an RNA, but it probably falls in the 0.1 to 0.05 range. With a threshold of 0.25 to distinguish active ODNs, the frequency of positives for the database is 0. 17. A calculation was performed to adjust for this discrepancy, by considering that the ratio of false positives to true positives will increase as the ratio of negative to positive ODN sequences in the database increases. This is because a false positive is a negative ODN that is mis-predicted as positive. Thus, a higher ratio of negatives leads to more mis-predictions (assuming the same rate). Correcting for this based on an estimated frequency of 0.10 for naturally occurring active ODNs, the above P⁺ numbers become 0.31 for Tu's method (G. C. Tu et al., supra) and 0.577 for the neural network of the present invention.

A web-based interface has been devised to the neural network predictions disclosed above. The interface provides for the entry of an RNA string and selection of how many resulting ODNs are displayed. The program then scans the neural network across the sequence string, stepping from left to right one base at a time, with a default ODN size of 20 nt. At each step, the ODN corresponding to that site is evaluated by the network. The results are stored in memory, and after all sites are evaluated, the results are sorted from best to worst predicted ODN. The network score (lower is better) is provided, together with an experimental probability value calculated by a logistic regression. The probability value gives a rough estimate of the probability that a given ODN will be active. However, there are still some unresolved issues regarding how to best cross validate the logistic regression values. So, for the time being the regression function is based on the take-one-out cross validation data, which in practice appear to provide somewhat low estimates of the probability of the ODN being active.

A user of this web-based interface would enter an RNA sequence into the web site, select the top n ODNs returned by the prediction, and then test them in the laboratory. The number n depends on resources, the need to find an extremely active ODN, and so on, but a reasonable number might be 10 or 20. For example, using take-one-out cross validation, the best 20 ODN sequences in the database were examined according to the neural network predictions shown in Table 1. Of the predicted best ten, 8 of them were in fact active, with active ODNs defined as reducing RNA expression to less that 0.25 of the control. Of the best twenty predicted, 14 were active, with two near misses (0.25 and 0.26). Even if the predictions were affected slightly by the lower incidence of positive sites in nature than in the database, these results are good enough that by testing only the top 2-3 predicted ODNs, a positive result is quite likely. Thus, this tool should greatly reduce the amount of laboratory time spent screening for active ODNs. Using the P⁺ of 0.57 from above, the savings should be at least five-fold in the number of ODNs that must be screened on average to find an active one. However, in reality the reduction in effort is likely to be greater if this approach of testing the ODNs in order of predicted efficacy is followed.

TABLE 1

| | Oligonucleotide | In vivo | Network | Regression |
|---|---|---|---|---|
| RNA | (SEQ ID NO:) | Activity | Prediction | Probability |
| TNF | 1 | 0.1 | −1.16 | 0.95 |
| PKC-alpha | 2 | 0.46 | −1.08 | 0.94 |
| TNF | 3 | 0.45 | −0.86 | 0.89 |
| TNF | 4 | 0.14 | −0.51 | 0.77 |
| TNF | 5 | 0.11 | −0.26 | 0.63 |
| VCAM | 6 | 0.09 | −0.24 | 0.62 |
| ICAM | 7 | 0 | −0.17 | 0.58 |
| MDR | 8 | 0 | −0.14 | 0.56 |
| ICAM | 9 | 0.1 | −0.12 | 0.54 |
| TNF | 10 | 0.2 | 0.02 | 0.45 |
| VCAM | 11 | 0.16 | 0.03 | 0.45 |
| VCAM | 12 | 0.26 | 0.05 | 0.43 |
| ICAM | 13 | 0.07 | 0.05 | 0.43 |
| IL-1 | 14 | 0.75 | 0.06 | 0.43 |
| TNF | 15 | 0.38 | 0.09 | 0.41 |
| VCAM | 16 | 0.1 | 0.1 | 0.4 |
| TNF | 17 | 0.06 | 0.1 | 0.4 |
| ICAM | 18 | 0.25 | 0.12 | 0.39 |
| TNF | 19 | 0.1 | 0.12 | 0.39 |
| VCAM | 20 | 0.21 | 0.13 | 0.38 |

| | | | |
|---|---|---|---|
| Average | 0.1995 | −0.1835 | 0.552 |

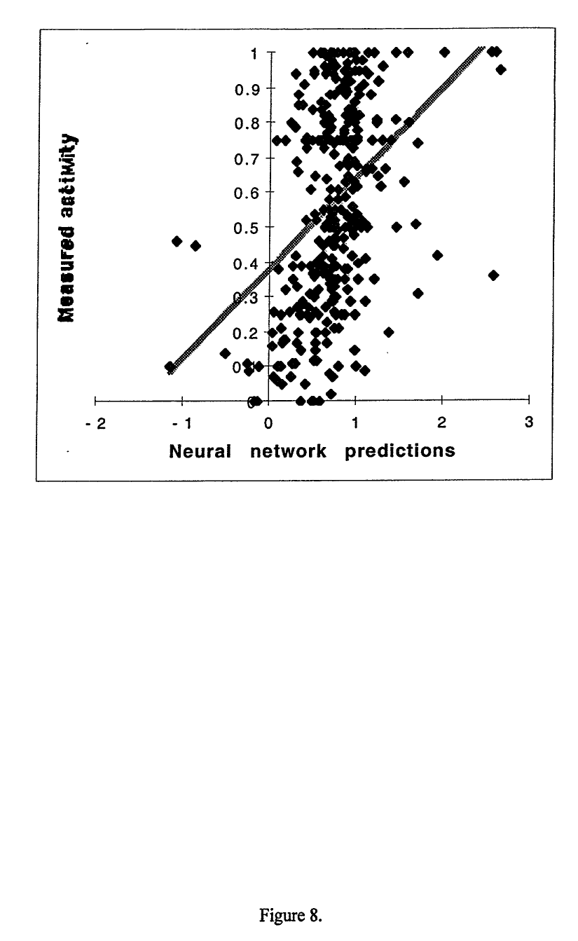

Performing a regression analysis on the take-one-out data for this network produced a fit with an R value of 0.38, and a significance of 1.9×10⁻¹³. This significance value indicates that it is highly unlikely these predictions were an accident of one particular experiment. The regression plot in FIG. 8 shows the correlation is best in the outlying regions, i.e., for those ODNs predicted most and least active. When the central region is considered, consisting of predictions in the range 0-1, the R value drops to 0.35 and significance to 1.0×10⁹.

The surprisingly good performance of these neural network predictions indicates that there must be one or more strong sequence-specific effects on antisense oligonucleotide action. The effects must be significant or they would not be recognizable within a database such as this, since it contains such a great deal of variability and noise. One possible explanation for the motif bias is RNase H sequence specificity at the double stranded region to which it binds, acting in addition to structural and energetic mechanisms. It is also possible that the ODN delivery process could exhibit motif-based biases. It is unlikely that non sequence-specific effects are at play, since all of the data used were collected utilizing control ODNs.

### EXAMPLE 1

In this example, an artificial neural network was prepared according to the following parameters: backpropagation with a momentum term, learning rate=0.025, momentum=0.05, c=0.1, dₘₐₓ=0.0, training for 450 cycles, log transform of target outputs as described above, linear activation function on the output node as described above, and a 40-4-1 layered architecture (i.e., Chi-40 network).

### EXAMPLE 2

In this example, an artificial neural network was prepared according to the following parameters: backpropagation with a momentum term, learning rate=0.2, momentum=0.1, c=0.2, dₘₐₓ=0.0, training for 1000 cycles, 3-way piecewise activation function for training output as described above, linear activation function on the output node as described above, and a 40-4-1 layered architecture (i.e., Chi-40 network).

The subject matter claimed is:

1. A method for predicting antisense activity of an oligonucleotide for down-regulating expression of a selected RNA comprising:
(a) developing an artificial neural network embodied on a computer-readable medium comprising
(i) constructing a database comprising sequence data of oligonucleotides tested in vivo for activity in down-regulating expression of RNAs and activity data corresponding to said sequence data,
(ii) providing an input layer containing a selected number of input nodes, optionally at least one hidden layer comprising a plurality of hidden nodes having full connectivity to said input nodes, and an output layer comprising at least one output node connected to said plurality of hidden nodes, if present, or to said input nodes,
(iii) mapping sequence motifs of a preselected length found in the sequence data contained in the database, entering counts for each of said sequence motifs in selected input nodes of the input layer, and entering the activity data correlated with said counts of said sequence motifs, and
(iv) training the artificial neural network having the counts entered in the input layer thereof such that the artificial neural network produces an output in the output layer upon entry of sequence motif counts, wherein said output comprises a measure of predicted activity correlated with sequence motif counts for a test oligonucleotide; and
(b) mapping sequence motifs of the preselected length present in a nucleotide sequence of a test oligonucleotide complementary to at least a portion of said selected RNA, determining counts of the mapped sequence motifs, and entering the counts of said sequence motifs present in the nucleotide sequence of said test oligonucleotide in the input layer of the artificial neural network; and
(c) obtaining output of the predicted antisense activity of the test oligonucleotide for down-regulating expression of said selected RNA.

2. The method of claim 1 wherein the sequence data in said database comprise sequence data compiled from published articles wherein each of said published articles reports results obtained with at least ten oligonucleotides and at least one mismatch or scrambled control oligonucleotide.

3. The method of claim 1 wherein said input layer comprises one input node per sequence motif.

4. The method of claim 1 wherein said input layer comprises only sequence motifs exhibiting a statistical correlation in their presence to oligonucleotide activity.

5. The method of claim 4 wherein a χ² test for significance is performed on the sequence motifs for all oligonucleotide sequences in the database, such sequence motifs are ranked from most to least significant, and the selected number of input nodes corresponds to a selected number of most significant sequence motifs, one input node per most significant sequence motif.

6. The method of claim 5 wherein said selected number of most significant sequence motifs is about 20 to about 80.

7. The method of claim 6 wherein said number of most significant sequence motifs is about 40.

8. The method of claim 1 wherein said at least one hidden layer comprises from about 4 to about 16 hidden nodes.

9. The method of claim 1 wherein said at least one hidden layer comprises about 4 hidden nodes.

10. The method of claim 1 wherein said output layer comprises one output node.

11. The method of claim 1 wherein said training the neural network further comprises using a back-propagation algorithm with a momentum term.

12. The method of claim 1 wherein said training the neural network further comprises using a back-propagation algorithm without a momentum term.

13. The method of claim 1 further comprising reporting accuracy of predicted antisense activity by ROC analysis.

14. The method of claim 1 further comprising assessing generalization of predicted antisense activity by minus 10% cross validation.

15. The method of claim 1 further comprising assessing generalization of predicted antisense activity by take-one-out cross-validation.

16. The method of claim 1 further comprising assessing generalization of predicted antisense activity by means of minus-one-RNA cross-validation.

17. The method of claim 1 wherein said counts of sequence motifs are entered as normalized data.

18. The method of claim 1 wherein antisense activity of oligonucleotides is entered using a binary threshold function with a cutoff in the range of about 0.01-0.50.

19. The method of claim 1 wherein discrimination of antisense activity of low-activity oligonucleotides is emphasized and antisense activity of high-activity oligonucleotides is de-emphasized.

20. The method of claim 1 further comprising combining the predicted antisense activity of the artificial neural network with a predicted antisense activity of at least one other artificial neural network.

21. The method of claim 1 further comprising combining the predicted antisense activity of the artificial neural network with an estimator of free-energy change associated with oligonucleotide-RNA duplex creation.

22. A method of making an artificial neural network, embodied on a computer-readable medium, for predicting antisense activity of oligonucleotides for down-regulating expression of a selected RNA comprising:
(a) constructing a database comprising sequence data of oligonucleotides tested in vivo for activity in down-regulating expression of RNAs and activity data corresponding to said sequence data;
(b) providing an input layer containing a selected number of input nodes, optionally at least one hidden layer comprising a plurality of hidden nodes having full connectivity to said input nodes, and an output layer comprising at least one output node connected to said plurality of hidden nodes, if present, or to said input nodes;
(c) mapping sequence motifs of a preselected length found in the sequence data contained in the database, entering counts for each of said sequence motifs in selected input nodes of the input layer, and entering the activity data correlated with said counts of said sequence motifs; and
(d) training the artificial neural network having the counts entered in the input layer thereof such that the artificial neural network produces an output in the output layer upon entry of sequence motif counts, wherein said output comprises a measure of predicted activity correlated with sequence motif counts for a test oligonucleotide.

23. The method of claim 22 wherein the sequence data in said database comprise sequence data compiled from published articles wherein each of said published articles reports results obtained with at least ten oligonucleotides and at least one mismatch or scrambled control oligonucleotide.

24. The method of claim 22 wherein said input layer comprises one input node per sequence motif.

25. The method of claim 22 wherein said input layer comprises only sequence motifs exhibiting a statistical correlation in their presence to oligonucleotide activity.

26. The method of claim 25 wherein a χ² test for significance is performed on the sequence motifs for all oligonucleotide sequences in the database, such sequence motifs are ranked from most to least significant, and the selected number of input nodes corresponds to a selected number of most significant sequence motifs, one input node per most significant sequence motif.

27. The method of claim 26 wherein said selected number of most significant sequence motifs is about 20 to about 80.

28. The method of claim 27 wherein said number of most significant sequence motifs is about 40.

29. The method of claim 22 wherein said at least one hidden layer comprises from about 4 to about 16 hidden nodes.

30. The method of claim 22 wherein said at least one hidden layer comprises 4 hidden nodes.

31. The method of claim 22 wherein said output layer comprises one output node.

32. The method of claim 22 wherein said training the neural network further comprises using a back-propagation algorithm with a momentum term.

33. The method of claim 22 wherein said training the neural network further comprises using a back-propagation algorithm without a momentum term.

34. The method of claim 22 further comprising reporting accuracy of predicted antisense activity by ROC analysis.

35. The method of claim 22 further comprising assessing generalization of predicted antisense activity by minus 10% cross validation.

36. The method of claim 22 further comprising assessing generalization of predicted antisense activity by take-one-out cross-validation.

37. The method of claim 22 further comprising assessing generalization of predicted antisense activity by means of minus-one-RNA cross-validation.

38. The method of claim 22 wherein said counts of sequence motifs are entered as normalized data.

39. The method of claim 22 wherein antisense activity of oligonucleotides is entered using a binary threshold function with a cutoff in the range of about 0.01-0.50.

40. The method of claim 22 wherein discrimination of antisense activity of low-activity oligonucleotides is emphasized and antisense activity of high-activity oligonucleotides is de-emphasized.

41. The method of claim 22 further comprising combining the predicted antisense activity of the artificial neural network with a predicted antisense activity of at least one other artificial neural network.

42. The method of claim 22 further comprising combining the predicted antisense activity of the artificial neural network with an estimator of free-energy change associated with oligonucleotide-RNA duplex creation.

43. An artificial neural network embodied on a computer-readable medium made by the method of claim 22.

44. An artificial neural network embodied on a computer-readable medium comprising:
(a) an input layer containing a selected number of input nodes;
(b) optionally at least one hidden layer comprising a plurality of hidden nodes having full connectivity to said input nodes; and
(c) an output layer comprising at least one output node connected to said plurality of hidden nodes, if present, or to said input nodes;
wherein sequence motifs of a preselected length found in a database comprising (i) sequence data of oligonucleotides tested in vivo for activity in down-regulating expression of RNAs and (ii) activity data corresponding to said sequence data are mapped and counts for each of said mapped sequence motifs are entered in selected input nodes of the input layer, and the activity data correlated with said counts of said sequence motifs are also entered in said selected input nodes of the input layer, and then the artificial neural network is trained such that the artificial neural network produces an output in the output layer upon entry of sequence motif counts, wherein said output comprises a measure of predicted activity correlated with sequence motif counts for a test oligonucleotide.

45. The artificial neural network of claim 44 wherein the sequence data in said database comprise sequence data compiled from published articles wherein each of said published articles reports results obtained with at least ten oligonucleotides and at least one mismatch or scrambled control oligonucleotide.

46. The artificial neural network of claim 44 wherein said input layer comprises one input node per sequence motif.

47. The artificial neural network of claim 44 wherein said input layer comprises only sequence motifs exhibiting a statistical correlation in their presence to oligonucleotide activity.

48. The artificial neural network of claim 47 wherein a χ² test for significance is performed on the sequence motifs for all oligonucleotide sequences in the database, such sequence motifs are ranked from most to least significant, and the selected number of input nodes corresponds to a selected number of most significant sequence motifs, one input node per most significant sequence motif.

49. The artificial neural network of claim 48 wherein said selected number of most significant sequence motifs in about 20 to about 80.

50. The artificial neural network of claim 49 wherein said number of most significant sequence motifs is about 40.

51. The artificial neural network of claim 44 wherein said at least one hidden layer comprises from about 4 to about 16 hidden nodes.

52. The artificial neural network of claim 44 wherein said at least one hidden layer comprises 4 hidden nodes.

53. The artificial neural network of claim 44 wherein said output layer comprises one output node.

54. The artificial neural network of claim 44 wherein said artificial neural network is trained using a back-propagation algorithm with a momentum term.

55. The artificial neural network of claim 44 wherein said artificial neural network is trained using a back-propagation algorithm without a momentum term.

56. The artificial neural network of claim 44 wherein accuracy of predicted antisense activity is reported by ROC analysis.

57. The artificial neural network of claim 44 wherein generalization of predicted antisense activity is assessed by minus 10% cross validation.

58. The artificial neural network of claim 44 wherein generalization of predicted antisense activity is assessed by take-one-out cross-validation.

59. The artificial neural network of claim 44 generalization of predicted antisense activity is assessed by means of minus-one-RNA cross-validation.

60. The artificial neural network of claim 44 wherein said counts of sequence motifs are entered as normalized data.

61. The artificial neural network of claim 44 wherein antisense activity of oligonucleotides is entered using a binary threshold function with a cutoff in the range of about 0.01-0.50.

62. The artificial neural network of claim 44 wherein discrimination of antisense activity of low-activity oligonucleotides is emphasized and antisense activity of high-activity oligonucleotides is de-emphasized.

63. The artificial neural network of claim 44 wherein the predicted antisense activity is combined with a predicted antisense activity of at least one other artificial neural network.

64. The artificial neural network of claim 44 wherein the predicted antisense activity is combined with an estimator of free-energy change associated with oligonucleotide-RNA duplex creation.
